(19) United States
(12) Patent Application Publication
(10) Pub. No.: US 2021/0338539 A1
(43) Pub. Date: Nov. 4, 2021

(54) LAMELLAR GEL BASED PERSONAL CARE COMPOSITIONS, PROCESS FOR PREPARING THE SAME AND METHOD OF USE THEREOF

(71) Applicant: ISP INVESTMENTS LLC, Wilmington, DE (US)

(72) Inventors: David C. STREULI, Wayne, NJ (US); Anthony LUSCHEN, Wayne, NJ (US); Hani M. FARES, Somerset, NJ (US); Rita Marie GUERRERO, Hillsborough, NJ (US)

(21) Appl. No.: 17/283,814

(22) PCT Filed: Sep. 23, 2019
(86) PCT No.: PCT/US19/52364

§ 371 Date: Apr. 8, 2021

Related U.S. Application Data
(60) Provisional application No. 62/734,779, filed on Sep. 21, 2018.

(51) Int. Cl. A61K 8/04; A61K 8/81; A61K 8/34; A61K 8/36; A61K 8/37; A61K 8/92; A61K 8/40; A61K 8/35; A61Q 17/04

(52) U.S. Cl. CPC A61K 8/042; A61K 8/8182; A61K 8/8152; A61K 8/8147; A61K 8/342; A61K 8/361; A61K 2800/5922; A61K 8/922; A61K 8/40; A61K 8/37; A61K 8/35; A61Q 17/04; A61K 8/375

(57) ABSTRACT

A personal care composition comprising i) 0.1 wt. % to 10 wt. % of a terpolymer of vinyl pyrrolidone/acrylic acid/lauryl methacrylate having a relative viscosity of at least 3.3 cps; and at least one lamellar gel. Other embodiments of this application disclose specific sunscreen compositions, methods for providing enhanced care and sensory benefits to skin of humans or animals and applying such a composition to the skin.

### FIELD OF THE INVENTION

The present application relates to a personal care composition, and, particularly, to lamellar gel based personal care composition. More specifically, the present application relates to a personal care composition comprising i) 0.1 wt. % to 10 wt. % of a terpolymer of vinyl pyrrolidone/acrylic acid/lauryl methacrylate having a relative viscosity of at least 3.3 cps; and ii) at least one lamellar gel. The present application also relates to methods of using the composition.

### BACKGROUND OF THE INVENTION

Water resistance is sought as one of the most desirable properties in the cosmetic/personal care industry. There have been considerable efforts in the cosmetic/personal care industry to provide personal care/cosmetic products having improved water resistance, particularly for sunscreen products, skin care products, and other cosmetic products. Water resistance of the skin care products especially of sunscreen products is considered a key element in addition to the other critical elements such as sun protection factor (SPF) and UVA protection. It is a well-known approach to use sunscreen products to limit the Ultra Violet (UV) radiation exposure to the skin. It has been observed that most commercially available sunscreen/skin care products suffer from poor water resistance, and the main reason for this poor water resistance of these products is the presence of surfactants. Most of these skin care products are available as emulsions comprising surfactants to reduce the surface tension between the hydrophilic and the hydrophobic ingredients of these products, however, use of such surfactants disadvantageously results in decreased water resistance of these products. Due to this decreased water resistance of these products, they tend to remove easily on exposure to water or sweat, thus, necessitating multiple applications of skin care product. Using emulsifiers that form lamellar gels to retain water resistance is the latest trend in manufacturing personal care compositions. Further, these emulsifiers offer multiple benefits to personal care formulations including improved moisturization, sensoriality and product stability.

Emulsifiers that form lamellar liquid gels are marketed by several cosmetic ingredient suppliers See, e.g., International Specialty Products, “Prolipid™ 141 For Skin Care,” pp. 3,4 (April 1999) (“Prolipid”); New Paradigm Technologies “Biobase™S: Product Information,” p. 1 (Undated) (“Biobase”); Seppic, “Montanov: Emulsifiers in Harmony with Nature”, p. 1 (July 2000) (“Montanov”). These lamellar liquid gels serve as barrier and water-retention properties of skin care products.

United States Patent Publication No. 20050238677 discloses a cosmetic or dermatological emulsifier system that forms a multi-lamellar liquid crystalline network comprising (i) a mixture of at least one first sucrose ester and at least one second sucrose ester and (ii) at least one solid fatty alcohol.

PCT Publication No. 2017197195A1 discloses a whipped or whippable formulation for topical application, such as, for example, sunscreen. The formulations disclosed have a desired sensory impact to a user.

In spite of continuous efforts in the personal care/cosmetic industry to develop skin care and/or sunscreen products having improved water resistance properties, there is still a need to envisage skin care compositions comprising improved sensory benefits are with respect to (a) providing protection from the harmful ultraviolet radiations of sun, (b) providing dry and powdery after feel to the skin, (c) providing decreased shine appearance, (d) providing enhanced water resistance of the sunscreen composition (e) providing positive appearance, (f) high integrity of shape, (g) visual compactness, (h) high spreadability, and/or (i) positive skin feel. Accordingly, it is an objective of the present application to provide a skin care composition having such improved properties.

### SUMMARY OF THE INVENTION

The present application provides a personal care composition comprising: i) 0.1 wt. % A to 10 wt. % of a terpolymer of vinyl pyrrolidone/acrylic acid/lauryl methacrylate having a relative viscosity of at least 3.3 cps; and ii) at least one lamellar gel. Also, disclosed is a method for providing enhanced care to skin of humans or animals by applying above said composition to the skin.

Yet another important aspect of the present application discloses a sunscreen composition comprising: i) 0.1 wt. % to 10 wt. % of a terpolymer of vinyl pyrrolidone/acrylic acid/lauryl methacrylate having a relative viscosity of at least 3.3 cps; ii) at least one lamellar gel; iii) 0.1 wt. % to 60 wt. % of at least one sunscreen agent; and iv) 0.1 wt. % to 20 wt. % of at least one cosmetically/dermatologically acceptable additive.

According to another important embodiment of the present application, the weight average molecular weight of the terpolymer employed in the skincare and sunscreen composition ranges from 150,000 to 300,000 Daltons.

According to another embodiment of the present application, the composition employs at least one cosmetically/dermatologically acceptable additive selected from the group consisting of diluents, emollients, humectants, thickeners, preservatives, coloring agents, sunscreen agents, moisturizing agents, film formers/waterproofing agents, bio-active ingredients, pharmaceutical ingredients, pH adjusting agents, chelating agents, rheology modifying agents, fragrance agents, plant extracts, absorbents, vitamins, photo stabilizing agents, sunscreen boosters, anti-oxidants, exfoliating agents, liquid carriers, waxes, conditioners, lubricants, anti-bacterial agents, anti-aging agents, insect repellants, and various combinations thereof.

Yet another embodiment of the present application discloses that the skin care and the sunscreen compositions of the present application can be formulated in the form of gels, balms, cream-in-gels, balm-in-gels, sticks, lotions, foams, foam creams, creams, moisturizers, sprays, ointments, mousses and a lamellar gel networks (LGNs), and wherein the pH range of these compositions are in the range of from about 2 to about 12.

In accordance with another embodiment of the present application, the skin care and sunscreen compositions of the present application exhibit (a) protection from the harmful ultraviolet radiations of sun, (b) dry and powdery after feel to the skin, (c) decreased shine appearance, (d) enhanced water resistance of the sunscreen composition (e) positive appearance, (f) high integrity of shape, (g) visual compactness, (h) high spreadability, and/or (i) positive skin feel to the skin after its application.

### DETAILED DESCRIPTION OF THE INVENTION

While this specification concludes with claims particularly pointing out and distinctly claiming that which is regarded as the invention, it is anticipated that the invention can be more readily understood through reading the following detailed description of the invention and study of the included examples

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

The term “about” can indicate a difference of 10 percent of the value specified. Numerical ranges as used herein are meant to include every number and subset of numbers enclosed within that range, whether particularly disclosed or not. Further, these numerical ranges should be construed as providing support for a claim directed to any number or subset of numbers in that range.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All references to singular characteristics or limitations of the present invention shall include the corresponding plural characteristic or limitation, and vice-versa, unless otherwise specified or clearly implied to the contrary by the context in which the reference is made.

As used herein, the words “preferred” or “preferably” and variants refer to embodiments of the invention that afford certain benefits, under certain circumstances. However, other embodiments can also be preferred, under the same or other circumstances. Furthermore, the recitation of one or more preferred embodiments does not imply that other embodiments are not useful, and is not intended to exclude other embodiments from the scope of the invention.

References herein to “one embodiment” or “one aspect” or “one version” or “one objective” of the invention include one or more such embodiment, aspect, version or objective, unless the context clearly dictates otherwise.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” can extend up to 100 or 1000 or more depending on the term to which it is attached. In addition, the quantities of 100/1000 are not to be considered limiting as lower or higher limits can also produce satisfactory results. In addition, the use of the term “at least one of X, Y, and Z” will be understood to include X alone, Y alone, and Z alone, as well as any combinations of X, Y, and Z. The use of ordinal number terminology (i.e., “first”, “second”, “third”, “fourth”, etc.) is solely for the purpose of differentiating between two or more items and, unless otherwise stated, is not meant to imply any sequence or order or importance to one item over another or any order of addition.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “or combinations thereof” as used herein refers to all permutations and combinations of the listed items preceding the term. For example, “A, BXn, BXn+1, or combinations thereof” is intended to include at least one of: A, BXn, BXn+1, ABXn, A BXn+1, BXnBXn+1, or ABXnBXn+1 and, if order is important in a particular context, also BXnA, BXn+1A, BXn+1BXn, BXn+1BXnA, BXnBXn+1A, ABXn+1BXn, BXnABXn+1, or BXn+1ABXn. Continuing with this example, expressly included are combinations that contain repeats of one or more item or term, such as BXnBXn, AAA, MBXn, BXnBXnBXn+1, AAABXnBXn+1BXn+1BXn+1BXn+1, BXn+1BXnBXnAAA, BXn+1A BXnABXnBXn, and so forth. The skilled artisan will understand that typically there is no limit on the number of items or terms in any combination, unless otherwise apparent from the context.

The term “polymer” refers to a large molecule comprising one or more types of monomer residues (repeating units) connected by covalent chemical bonds. Polymers can be further derivatized, crosslinked, grafted or end-capped. By this definition, polymer encompasses compounds wherein the number of monomer units can range from very few, which more commonly can be called as oligomers, to very many. Non-limiting examples of polymers include homopolymers, and non-homopolymers such as copolymers, terpolymers, tetrapolymers and the higher analogues. The polymer can have a random, block, and/or alternating architecture.

The term “homopolymer” refers to a polymer that consists essentially of a single monomer type. The term “non-homopolymer” refers to a polymer that comprises more than one monomer types. The term “copolymer” refers to a non-homopolymer that comprises two different monomer types. The term “terpolymer” refers to a non-homopolymer that comprises three different monomer types. The term “branched” refers to any non-linear molecular structure. The term includes both branched and hyper-branched structures.

The term “keratin substrate” as used herein includes skin, nails and “keratin fibers”, and wherein the “keratin fibers” means hair on head, eyelashes, eyebrows and other mammalian bodily hair.

The terms “personal care composition” and “cosmetics” refer to compositions intended for use on or in the human body, such as skin, sun, hair, oral, cosmetic, and preservative compositions, including those to alter the color and appearance of the skin and hair.

What is described herein is a personal care composition comprising: i) 0.1 wt. % to 10 wt. % of a terpolymer of vinyl pyrrolidone/acrylic acid/lauryl methacrylate having a relative viscosity of at least 3.3 cps; and ii) at least one lamellar gel. Also, disclosed is a method for providing enhanced care to skin of humans or animals by applying the above said composition to the skin.

Further, the compositions of the present application feature a terpolymer having relative viscosity of at least at least 3.3 cps. The use of terpolymer having relative viscosity of at least 3.3 cps allows reduced levels or complete removal of surfactants from the compositions. The terpolymer as used in the compositions of the present application successfully stabilizes the surface tension between the continuous external phase and the discontinuous internal phase. In accordance with one of the embodiments of the present application, the terpolymer has relative viscosity in the range of from 3.3 to 4.0 cps. Further, it is contemplated to employ terpolymers having relative viscosity ranges of from 3.3 to 6.0 cps.

The relative viscosity of the terpolymer of the present application is measured through a K-value determination by capillary viscometry. In this method the relative viscosity of the 1% solution of the terpolymer is determined by dividing the flow time through a capillary viscometer of the 1% terpolymer solution, by the flow time of a 0.25N NaOH, 0.20M LiNO₃; aqueous solution. K-value is calculated from the relative viscosity using the Fikentscher equation.

The relative viscosity, ηᵣₑₗ, is calculated using Equation 1

$${η}_{rel} = \frac{flow time of solution}{flow time of solvent} \qquad ( 1 )$$

The K-Value can be calculated using Equation 2

$$K = \frac{\sqrt{300 c log {η}_{rel} \div {( c + 1.5 c log {η}_{rel} )}^{2}} + 1.5 c log {η}_{rel} - c}{0. .15 c \div {0.003}_{c}^{2}} \qquad ( 2 )$$

According to yet another embodiment of the present application, the weight average molecular weight of the terpolymer ranges from 150,000 to 300,000 Daltons. Other weight average molecular weight of terpolymer ranges, include but are not limited to, from 150,000 to 175,000 Daltons, from 175,000 to 200,000 Daltons, from 200,000 to 225,000 Daltons, from 225,000 to 250,000 Daltons, from 250,000 to 275,000 Daltons, or from 275,000 to 300,000 Daltons. Other important and specific weight average molecular weight of terpolymer range is from 180,000 to 220,000 Daltons, and from 205,000 to 215,000 Daltons, 207,000 Daltons, 208,000 Daltons, 209,000 Daltons, 210,000 Daltons, 211,000 Daltons, or 212,000 Daltons.

The terpolymers which may be used in accordance with the invention are preferably chosen from polymers containing 40 to 70% vinylpyrrolidone, 15-40% unsaturated carboxylic acid and 5 to 20% of alkyl methacrylate, having 8-18 carbon atoms.

One embodiment of the present application employs 0.1 wt. % to 10 wt. % of a terpolymer of vinyl pyrrolidone/acrylic acid/lauryl methacrylate having a relative viscosity of at least 3.3 cps, and wherein, other ranges of terpolymer, include but are not limited to, 0.1 wt. % to 1 wt. %, 1 wt. % to 2 wt. %, 2 wt. % to 3 wt. %, 3 wt. % to 4 wt. % 4, 4 wt. % to 5 wt. %, 5 wt. % to 6 wt. %, 6 wt. % to 7 wt. %, 7 wt. % to 8 wt. %, 8 wt. % to 9 wt. %, or 9 wt. % to 10 wt. %

According to one of embodiment of the present application, the lamellar gel is a blend of (a) one or more fatty alcohols; (b) one or more fatty acids and/or their esters; (c) optionally one or more low molecular weight alcohols; (d) optionally esterified oils; and (e) optionally cationic swellants.

In accordance with yet another embodiment of the present application, the lamellar gel is a blend of: (a) one or more C₈-C₂₄ fatty alcohols; (b) one or more C₈-C₂₄ fatty acids and/or their esters; (c) optionally a C₂-C₁₀ low molecular weight alcohols; (d) optionally esterified oils; and (e) optionally cationic swellants.

According to another embodiment of the present application, the C₈-C₂₄ fatty alcohol is selected from the group consisting of behenyl alcohol, cetyl alcohol, stearyl alcohol, lauryl alcohol, myristyl alcohol and a mixture thereof.

According to another embodiment of the present application, the C₈-C₂₄ fatty acid is selected from the group consisting of stearic acid, palmitic acid and a mixture thereof.

According to one more embodiment of the present application, the C₈-C₂₄ fatty acid ester is selected from the group consisting of glyceryl stearate, sorbitan monostearate, ricinoleyl monomaleate, PEG-100 stearate, triglycerides and a mixture thereof.

In accordance with yet another embodiment of the present application, the C₂-C₁₀ low molecular weight alcohol is selected from the group consisting of propylene glycol, butylene glycol, isopropyl alcohol and a mixture thereof.

In accordance with one more embodiments of the present application, the lamellar gel is selected from a group consisting of Prolipid™ 141 and Prolipid™ 151. Examples of lamellar gels are described in U.S. Pat. Nos. 5,849,315 and 6,368,607.

One embodiment of the present application employs the lamellar gel in an amount of about 1.00% w/w to about 20.00% w/w of the personal care composition, and wherein, other ranges of the lamellar gel, include but are not limited to, 0.1 wt. % to 1 wt. %, 1 wt. % to 2 wt. %, 2 wt. % to 3 wt. %, 3 wt. % to 4 wt. %, 4 wt. % to 5 wt. %, 5 wt. % to 6 wt. %, 6 wt. % to 7 wt. %, 7 wt. % to 8 wt. %, 8 wt. % to 9 wt. %, 9 wt. % to 10 wt. %, 10 wt. % to 11 wt. %, 11 wt. % to 12 wt. %, 12 wt. % to 13 wt. %, 13 wt. % to 14 wt. %, 14 wt. % to 15 wt. %, 15 wt. % to 16 wt. %, 16 wt. % to 17 wt. %, 17 wt. % to 18 wt. %, 18 wt. % to 19 wt. %, or 19 wt. % to 20 wt. %.

In accordance with yet another embodiment of the present application, the lamellar gel comprises at least one esterified oil.

According to one more embodiments of the present application, the esterified oil is a maleated soybean oil.

According to one more embodiments of the present application, the lamellar gel comprises at least one cationic swellant.

According to one more embodiments of the present application, the cationic swellant is selected from the group consisting of a saturated fatty acid adduct of amidopropyldimethyl-2-hydroxy ethyl ammonium chloride and lecithin.

According to another embodiment of the present application, the personal care composition further comprises at least one propellant selected from the group consisting of CO₂, propane, butane, isobutane, dimethyl ether, nitrous oxide, nitrogen, oxygen, air, isopentane, hydrofluroolefin, and a mixture thereof.

One embodiment of the present application employs the propellant in an amount of about 1.00% w/w to about 20.00% w/w of the personal care composition, and wherein, other ranges of the propellant, include but are not limited to, 0.1 wt. % to 1 wt. %, 1 wt. % to 2 wt. % 2 wt. % to 3 wt. %, 3 wt. % to 4 wt. %, 4 wt. % to 5 wt. %, 5 wt. % to 6 wt. %, 6 wt. % to 7 wt. %, 7 wt. % to 8 wt. %, 8 wt. % to 9 wt. %, 9 wt. % to 10 wt. %, 10 wt. % to 11 wt. %, 11 wt. % to 12 wt. %, 12 wt. % to 13 wt. %, 13 wt. % to 14 wt. %, 14 wt. % to 15 wt. %, 15 wt. % to 16 wt. %, 16 wt. % to 17 wt. %, 17 wt. % to 18 wt. %, 18 wt. % to 19 wt. %, or 19 wt. % to 20 wt. %.

As used herein, the term “cosmetically/dermatologically acceptable excipient” means any ingredient/compound or mixture of ingredients/compounds or compositions that are typically employed to produce other desirable effects in skin care/sunscreen compositions. The preferred cosmetically/dermatologically acceptable excipients include but are not limited to diluents, preservatives, antioxidants, chelating agents, sunscreen agents/compounds/blockers, proteins, amino acids, vitamins, dyes, hair coloring agents, plant extracts, plant derivatives, plant tissue extracts, plant seed extracts, plant oils, botanicals, botanical extracts, humectants, fragrances, perfumes, oils, emollients, lubricants, butters, penetrants, thickeners, viscosity modifiers, thickeners, polymers, resins, hair fixatives, film formers, surfactants, detergents, emulsifiers, opacifying agents, volatiles, liquid vehicles, carriers, salts, pH adjusting agents, neutralizing agents, buffers, hair conditioning agents, anti-static agents, anti-frizz agents, anti-dandruff agents, hair waving agents, hair straightening agents, relaxers, absorbents, fatty substances, gelling agents, moisturizers, hydrophilic or lipophilic active agents, preserving agents, fillers, dyestuffs, reducing agents, cosmetic oils, perfumes, liquid vehicles, solvents, silicones, moisturizing agents, waterproofing agents, bio-active ingredients, pharmaceutical ingredients, rheology modifying agents, photo stabilizing agents, conditioners, exfoliating agents, anti-bacterial agents, waxes, insect repellants, anti-aging agents, anti-oxidants and combinations thereof.

According to another embodiment of the present application, the insect repellant is selected from the group consisting of N,N-diethyl m-toluamide (DEET), p-menthane-3,8-diol, permethrin, allethrin, piperonyl butoxide, lemongrass oil, citronella oil, eucalyptus oil, camphor, geranium oil, ethyl hexanediol, ethyl butylacetylaminopropionate, and hydroxyethyl-isobutyl-piperidine.

Example of various UV-A or UV-B based sunscreens compounds include, but are not limited to, p-aminobenzoic acid and its derivatives, anthranilates, benzophenones, camphor derivatives, cinnamic derivatives, dibenzoyl methanes, β,β-diphenylacrylate derivatives, salicylic derivatives, triazine derivatives, benzimidazole compounds, his-benzoazolyl derivatives, methylene bis-(hydroxyphenylbenzotriazole) compounds, sunscreen polymers, silicones, and mixtures thereof.

Examples of physical sun blockers include, but are not limited to, cerium oxides, chromium oxides, cobalt oxides, iron oxides, red petrolatum, silicone-treated titanium dioxides, titanium dioxides, zinc oxides, and/or zirconium oxides and mixtures thereof.

Suitable sunscreen agents for preparing the sunscreen compositions of the present application include, but are not limited to, methoxydibenzoylmethane; octyl salicylate; pentyl dimethyl PABA; octyl dimethyl PABA; benzophenone-1; benzophenone-6; 2-(2H-benzotriazole-2-yl)-4,6-di-tert-pentylphenol; ethyl-2-cyano-3,3-diphenylacrylate; homomethyl salicylate (homosalate); bis-ethylhexyloxyphenol methoxyphenyl triazine; methyl-(1,2,2,6,6-pentamethyl-4-piperidyl)-sebacate; 2-(2H-benzotriazole-2-yl)-4-methylphenol; diethylhexyl butamido triazone; amyl dimethyl PABA; 4,6-bis(octylthiomethyl)-o-cresol; red petroleum; ethylhexyl triazone; octocrylene; isoamyl-p-methoxycinnamate; drometrizole; titanium dioxide; 2,4-di-tert-butyl-6-(5-chloro-2H-benzotriazole-2-yl)-phenol; 2-hydroxy-4-octyloxybenzophenone; benzophenone-2; diisopropyl methylcinnamate; PEG-25 PABA; 2-(1,1-dimethylethyl)-6-[[3-(1,1-demethylethyl)-2-hydroxy-5-methylphenyl[methyl-4-methylphenyl acrylate; drometrizole trisiloxane; menthyl anthranilate; butyl methoxydibenzoylmethane; 2-ethoxyethyl p-methoxycinnamate; benzylidene camphor sulfonic acid; dimethoxyphenyl-[1-(3,4)]-4,4-dimethyl 1,3-pentanedione; zinc oxide; N,N′-hexane-1,6-diylbis[3-(3,5-di-tert-butyl-4-hydroxyphenylpropionamide)]; pentaerythritol tetrakis-[3-(3,5-di-tert-butyl-4-hydroxyphenyl)propionate]; 2,6-di-tert-butyl-4-[4,6-bis(octylthio)-1,3,5-triazin-2-ylamino]phenol; 2-(2H-benzotriazole-2-yl)-4,6-bis(1-methyl-1-phenylethyl)phenol; trolamine salicylate; diethylanolamine p-methoxycinnamate; polysilicone-15; 4-methylbenzylidene camphor; bisoctrizole; N-phenyl-benzenamine; reaction products with 2,4,4-trimethylpentene; sulisobenzone; (2-ethylhexyl)-2-cyano-3,3-diphenylacrylate; digalloyl trioleate; polyacrylamido methylbenzylidene camphor; glyceryl ethylhexanoate dimethoxycinnamate; 1,3-bis-[(2′-cyano-3′,3-diphenylacryloyl)oxy]-2,2-bis-[(2′-cyano-bis-(2,2,6,6-tetramethyl-4-piperidyl)-sebacate; benzophenone-5; 1,3,5-tris(3,5-di-tert-butyl-4-hydroxybenzyl)-1,3,5-triazone-2,4,6(1H,3H,5H)-trione; hexamethylendiamine; benzophenone-8; ethyl-4-bis(hydroxypropyl)aminobenzoate; 6-tert-butyl-2-(5-chloro-2H-benzotriazole-2-yl)-4-methylphenol; p-aminobenzoic acid; 3,3′,3″,5,5′,5″-hexa-tert-butyl-a-a′-a″-(mesitylene-2,4,6-triyl)tri-p-cresol; lawsone with dihydroxyacetone; benzophenone-9; benzophenone-4; ethylhexyl dimethoxy benzylidene dioxoimidazoline propionate; N,N′-bisformyl-N,N′-bis-(2,2,6,6-tetramethyl-4-piperidinyl)-; 3-benzylidene camphor; terephthalylidene dicamphor sulfonic acid; camphor benzalkonium methosulfate; bisdisulizole disodium; etocrylene; ferulic acid; 2-(2H-benzotriazole-2-yl)-4-(1,1,3,3-tetramethylbutyl)-phenol; 4,6-bis(dodecylthiomethyl)-o-cresol; [3-2-glucopyranoxy propyl hydroxy benzophenone; phenylbenzimidazole sulfonic acid; benzophenone-3; diethylamine hydroxybenzoyl hexylbenzoate; 3′,3′-diphenylacryloyloxymethyl-propane; ethylhexyl p-methoxycinnamate, and blends thereof.

According to yet another embodiment, the sunscreen composition of present application has a sun protection factor (SPF) of at least about 10. Other non-limiting ranges of SPF of the sunscreen composition, include but are not limited to, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30. at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 43, at least 44, at least 45, at least 46, at least 47, at least 48, at least 49, at least 50, at least 51, at least 52, at least 53, at least 54, at least 55, at least 56, at least 57, at least 58, at least 59, or at least 60.

Suitable emulsifiers include the following classes of ethers and esters: ethers of polyglycols and of fatty alcohols, esters of polyglycols and of fatty acids, ethers of polyglycols and of fatty alcohols which are glycosylated, esters of polyglycols and of fatty acids which are glycosylated, ethers of C₁₂₋₃₀ alcohols and of glycerol or of polyglycerol, esters of C₁₂₋₃₀ fatty acids and of glycerol or of polyglycerol, ethers of oxyalkylene-modified C₁₂₋₃₀ alcohols and of glycerol or polyglycerol, ethers of C₁₂₋₃₀ fatty alcohols comprising and of sucrose or of glucose, esters of sucrose and of C₁₂₋₃₀ fatty acids, esters of pentaerythritol and of C₁₂₋₃₀ fatty acids, esters of sorbitol and/or of sorbitan and of C₁₂₋₃₀ fatty acids, ethers of sorbitol and/or of sorbitan and of alkoxylated sorbitan, ethers of polyglycols and of cholesterol, esters of C₁₂₋₃₀ fatty acids and of alkoxylated ethers of sorbitol and/or sorbitan, and combinations thereof. Linear or branched type silicone emulsifiers can also be used.

The skin care and sunscreen composition of present application can be preserved by adding preservatives to the compositions. Such preservatives can be selected from, but are not limited to triazoles, imidazoles, naphthalene derivatives, benzimidazoles, morphline derivatives, dithiocarbamates, benzisothiazoles, benzamides, boron compounds, formaldehyde donors, isothiazolones, thiocyanates, quaternary ammonium compounds, iodine derivates, phenol derivatives, micobicides, pyridines, dialkylthiocarbamates, nitriles, parabens, alkyl parabens and salts thereof. The range of preservative employed is in the range of from about 0.01 wt. % to about 10 wt. %.

The preferred fatty substance-based additive/excipient for the present application includes fatty alcohols, natural and synthetic waxes, ceramides, mineral oils, vegetable oils, animal oils, synthetic oils. The other preferred fatty substances include isododecane, hydrogenated polyisobutene, squalane, isononyl isononanoate, cyclotetra- and -pentadimethicones, phenyltrimethicone, ethylene homopolymers, ethoxylated fats and oils, fluoroalkanes, seracite, shea butter, arachidyl propionate alone or in combination. For the definition of waxes, mention can be made, for example, of P. D. Dorgan, Drug and Cosmetic Industry, December 1983, pp. 30-33.

Moisturizers employed in the present application, include but are not limited to, glycols, glycerols, propylene glycol, diethylene glycol monoethyl ether, sorbitol, sodium salt of pyroglutamic acid, glycerol, glycerol derivatives, glycerin, trehalose, sorbitol, maltitol, dipropylene glycol, 1,3-butylene glycol, sodium hyaluronate, and the like. Examples of humectants which can be incorporated into a product of the present application are glycerin, propylene glycol, polypropylene glycol, polyethylene glycol, lactic acid, sodium lactate, pyrrolidone carboxylic acid, urea, phospholipids, collagen, elastin, ceramides, lecithin sorbitol, PEG-4, and mixtures thereof. Additional suitable moisturizers are polymeric moisturizers that are water soluble and/or water swellable in nature. Polysaccharides such as hyaluronic acid, chitosan can also be employed along with moisturizers of the present application as binder to enhance their property. The range of moisturizer is from about 0.1 wt. % to about 10 wt. %.

The suitable solvent of the present application can consist of water, a cosmetically/dermatologically acceptable solvent, or a blend of water and a cosmetically/dermatologically acceptable solvent, such as a lower alcohol composed of C₁ to C₄, such as ethanol, isopropanol, t-butanol, n-butanol, alkylene glycols such as propylene glycol, and glycol ethers. The most preferred solvents of the present application, include but are not limited to, water, ethanol and/or iso-propanol. It is contemplated to employ other suitable solvents for preparing products of the present application which include but are not limited to, linear and branched C₁-C₆ alcohols, such as ethanol, propanol, isopropanol, butanol, hexanol, and mixtures thereof; aromatic alcohols, such as benzyl alcohol, cycloaliphatic alcohols, such as cyclohexanol, and the like; saturated C₁₂-C₃₀ fatty alcohol, such as lauryl alcohol, myristyl alcohol, cetyl alcohol, stearyl alcohol, behenyl alcohol, and the like. Non-limiting examples of polyols include polyhydroxy alcohols, such as glycerin, propylene glycol, butylene glycol, hexylene glycol, C₂-C₄ alkoxylated alcohols and C₂-C₄ alkoxylated polyols, such as ethoxylated, propoxylated, and butoxylated ethers of alcohols, diols, and polyols having about 2 to about 30 carbon atoms and 1 to about 40 alkoxy units, polypropylene glycol, polybutylene glycol, and the like. Non-limiting examples of non-aqueous auxiliary solvents include silicones, and silicone derivatives, such as cyclomethicone, and the like, aliphatic solvents such as cyclohexane and heptane, ketones such as acetone and methyl ethyl ketone, and mixtures thereof; ethers such as diethyl ether, dimethoxymethane, and mixtures thereof, natural and synthetic oils and waxes, such as vegetable oils, plant oils, animal oils, essential oils, mineral oils, C₇-C₄₀ isoparaffins, alkyl carboxylic esters, such as ethyl acetate, amyl acetate, ethyl lactate, and the like, jojoba oil, shark liver oil, and the like.

The alkaline pH adjusting agents can be selected from alkali metal hydroxides, for example, sodium hydroxide, and potassium hydroxide; ammonium hydroxide; organic bases, such as triethanolamine, diisopropylamine, dodecylamine, diisopropanolamine, aminomethyl propanol, cocamine, oleamine, morpholine, triamylamine, triethylamine, tromethamine (2-amino-7-hydroxymethyl)-1,3-propanediol), and tetrakis(hydroxypropyl)ethylenediamine; and alkali metal salts of inorganic acids, such as sodium borate (borax), sodium phosphate, sodium pyrophosphate, and the like, and mixtures thereof. Acidic pH adjusting agents can be organic acids, including amino acids, and inorganic mineral acids. Non-limiting examples of acidic pH adjusting agents include acetic acid, citric acid, fumaric acid, glutamic acid, glycolic acid, hydrochloric acid, lactic acid, nitric acid, phosphoric acid, sodium bisulfate, sulfuric acid, tartaric acid, and the like, and mixtures thereof. The desired pH of the personal care composition is in the range of from about 2 to about 13, and in some embodiment, it is preferably between about 4 to about 8. The utility levels of the pH modifying agent can be present in an effective amount required to achieve the desired pH level.

The coloring agents, colorants or dyes used herein include natural foods colors and dyes suitable for food, drug and cosmetic applications. These colorants are also known as FD & C, and D&C dyes and lakes and are preferably water-soluble in nature. A full recitation of all FD&C and D&C dyes and their corresponding chemical structures can be found in the Kirk-Othmer Encyclopedia of Chemical Technology, Volume 5, pages 857-884, which text is accordingly incorporated herein by reference. These coloring agents can be incorporated in an amount up to about 3%, more particularly up to about 2%, and in some cases less than about 1% by weight of the personal care compositions.

A perfume or fragrance obtained from natural or synthetic source can be employed in the present skin/sunscreen composition. The fragrance can be used along with a suitable solvent, diluents or carrier. Fragrances can be added in any conventionally known method, for example, admixing to a composition or blending with other ingredients used to form a composition, in amounts which are found to be useful to increase or impart the desired scent characteristics to the compositions. Fragrances for the present application can be one or more selected from the following non-limiting group of compounds such as essential oils, absolutes, resinoids, resins, concretes, hydrocarbons, alcohols, aldehydes, ketones, ethers, acids, esters, acetals, ketals, nitriles, including saturated and unsaturated compounds and aliphatic, carbocyclic and heterocyclic compounds.

The compositions of the invention may contain a sequestering or chelating agent. The term “sequestering agent” or “chelating agent” as used herein relates to a compound which is capable of bonding or complexing a metal ion between two or more atoms of the compound, thereby neutralizing or controlling harmful effects of such metal ions. Holding or bonding of a metal ion is through combination of one or more different types of bonds including coordination and/or ionic bonds. Further, the information on sequestering and chelating agents that are considered for the present application is duly disclosed in T. E. Furia, CRC Handbook of Food Additives, 2^{nd} Edition, pp. 271-294 (1972), and M. S. Peterson and A. M. Johnson (Eds.), Encyclopedia of Food Science, pp. 694-699 (1978) are incorporated herein by reference in its entirety.

The compositions of the invention may contain a thickener and/or viscosifiers. Non-limiting examples of suitable thickeners and/or viscosifiers include: Acetamide MEA; acrylamide/ethalkonium chloride acrylate copolymer; acrylamide/ethyltrimonium chloride acrylate/ethalkonium chloride acrylate copolymer; acrylamides copolymer; acrylamide/sodium acrylate copolymer; acrylamide/sodium acryloyldimethyltaurate copolymer; acrylates/acetoacetoxyethyl methacrylate copolymer; acrylates/beheneth-25 methacrylate copolymer; acrylates/C₁₀-C₃₀ alkyl acrylate crosspolymer; acrylates/ceteth-20 itaconate copolymer; acrylates/ceteth-20 methacrylate copolymer; acrylates/laureth-25 methacrylate copolymer; acrylates/palmeth-25 acrylate copolymer; acrylates/palmeth-25 itaconate copolymer; acrylates/steareth-50 acrylate copolymer; acrylates/steareth-20 itaconate copolymer; acrylates/steareth-20 methacrylate copolymer; acrylates/stearyl methacrylate copolymer; acrylates/vinyl isodecanoate crosspolymer; acrylic acid/acrylonitrogens copolymer; adipic acid/methyl DEA crosspolymer; agar; agarose; alcaligenes polysaccharides; algin; alginic acid; almondamide DEA; almondamidopropyl betaine; aluminum/magnesium hydroxide stearate; ammonium acrylates/acrylonitrogens copolymer; ammonium acrylates copolymer; ammonium acryloyldimethyhaurate/vinyl formamide copolymer; ammonium acryloyldimethyltaurate/VP copolymer; ammonium alginate; ammonium chloride; ammonium polyacryloyldimethyl taurate; ammonium sulfate; amylopectin; apricotamide DEA; apricotamidopropyl betaine; arachidyl alcohol; arachidyl glycol; Arachis hypogaea (peanut) flour; ascorbyl methylsilanol pectinate; astragalus gummifer gum; attapulgite; Avena sativa (oat) kernel flour; avocadamide DEA; avocadamidopropyl betaine; azelamide MEA; babassuamide DEA; babassuamide MEA; babassuamidopropyl betaine; behenamide DEA; behenamide MEA; behenamidopropyl betaine; behenyl betaine; bentonite; butoxy chitosan; caesalpinia spinosa gum; calcium alginate; calcium carboxymethyl cellulose; calcium carrageenan; calcium chloride; calcium potassium carbomer; calcium starch octenylsuccinate; C20-40 alkyl stearate; canolamidopropyl betaine; capramide DEA; capryl/capramidopropyl betaine; carbomer; carboxybutyl chitosan; carboxymethyl cellulose acetate butyrate; carboxymethyl chitin; carboxymethyl chitosan; carboxymethyl dextran; carboxymethyl hydroxyethylcellulose; carboxymethyl hydroxypropyl guar; carnitine; cellulose acetate propionate carboxylate; cellulose gum; Ceratonia siliqua gum; cetearyl alcohol; cetyl alcohol; cetyl babassuate; cetyl betaine; cetyl glycol; cetyl hydroxyethylcellulose; chimyl alcohol; cholesterol/HDI/pullulan copolymer; cholesteryl hexyl dicarbamate pullulan; citrus aurantium dulcis (orange) peel extract; cocamide DEA; cocamide MEA; cocamide MIPA; cocamidoethyl betaine; cocamidopropyl betaine; cocamidopropyl hydroxysultaine; coco-betaine; coco-hydroxysultaine; coconut alcohol; coco/oleamidopropyl betaine; coco-Sultaine; cocoyl sarcosinamide DEA; cornamide/cocamide DEA; cornamide DEA; croscarmellose; cross-linked bacillus/glucose/sodium glutamate ferment; cyamopsis tetragonoloba (guar) gum; decyl alcohol; decyl betaine; dehydroxanthan gum; dextrin; dibenzylidene sorbitol; diethanolaminooleamide DEA; diglycol/CHDM/isophthalates/SIP copolymer; dihydroabietyl behenate; dihydrogenated tallow benzylmonium hectorite; dihydroxyaluminum aminoacetate; dimethicone/PEG-10 crosspolymer; dimethicone/PEG-15 crosspolymer; dimethicone propyl PG-betaine; dimethylacrylamide/acrylic acid/polystyrene ethyl methacrylate copolymer; dimethylacrylamide/sodium acryloyldimethyltaurate crosspolymer; disteareth-100 IPDI; DMAPA acrylates/acrylic acid/acrylonitrogens copolymer; erucamidopropyl hydroxysultaine; ethylene/sodium acrylate copolymer; gelatin; gellan gum; glyceryl alginate; glycine soja (soybean) flour; guar hydroxypropyltrimonium chloride; hectorite; hyaluronic acid; hydrated silica; hydrogenated potato starch; hydrogenated tallow; hydrogenated tallowamide DEA; hydrogenated tallow betaine; hydroxybutyl methylcellulose; hydroxyethyl acrylate/sodium acryloyldimethyl taurate copolymer; hydroxyethylcellulose; hydroxyethyl chitosan; hydroxyethyl ethylcellulose; hydroxyethyl stearamide-MIPA; hydroxylauryl/hydroxymyristyl betaine; hydroxypropylcellulose; hydroxypropyl chitosan; hydroxypropyl ethylenediamine carbomer; hydroxypropyl guar; hydroxypropyl methylcellulose; hydroxypropyl methylcellulose stearoxy ether; hydroxypropyl starch; hydroxypropyl starch phosphate; hydroxypropyl xanthan gum; hydroxystearamide MEA; isobutylene/sodium maleate copolymer; isostearamide DEA; isostearamide MEA; isostearamide mIPA; isostearamidopropyl betaine; lactamide MEA; lanolinamide DEA; lauramide DEA; lauramide MEA; lauramide MIPA; lauramide/myristamide DEA; lauramidopropyl betaine; lauramidopropyl hydroxysultaine; laurimino bispropanediol; lauryl alcohol; lauryl betaine; lauryl hydroxysultaine; lauryl/myristyl glycol hydroxypropyl ether; lauryl sultaine; lecithinamide DEA; linoleamide DEA; linoleamide MEA; linoleamide MIPA; lithium magnesium silicate; lithium magnesium sodium silicate; macrocystis pyrifera (kelp); magnesium alginate; magnesium/aluminum/hydroxide/carbonate; magnesium aluminum silicate; magnesium silicate; magnesium trisilicate; methoxy PEG-22/dodecyl glycol copolymer; methylcellulose; methyl ethylcellulose; methyl hydroxyethylcellulose; microcrystalline cellulose; milkamidopropyl betaine; minkamide DEA; minkamidopropyl betaine; MIPA-myristate; montmorillonite; Moroccan lava clay; myristamide DEA; myristamide MEA; myristamide MIPA; myristamidopropyl betaine; myristamidopropyl hydroxysultaine; myristyl alcohol; myristyl betaine; natto gum; nonoxynyl hydroxyethylcellulose; oatamide MEA; oatamidopropyl betaine; octacosanyl glycol isostearate; octadecene/MA copolymer; oleamide DEA; oleamide MEA; oleamide MIPA; oleamidopropyl betaine; oleamidopropyl hydroxysultaine; oleyl betaine; olivamide DEA; olivamidopropyl betaine; oliveamide MEA; palmamide DEA; palmamide MEA; palmamide MIPA; palmamidopropyl betaine; palmitamide DEA; palmitamide MEA; palmitamidopropyl betaine; palm kernel alcohol; palm kernelamide DEA; palm kernelamide MEA; palm kernelamide MIPA; palm kernelamidopropyl betaine; peanutamide MEA; peanutamide MIPA; pectin; PEG-800; PEG-crosspolymer; PEG-150/decyl alcohol/SMDI copolymer; PEG-175 diisostearate; PEG-190 distearate; PEG-15 glyceryl tristearate; PEG-140 glyceryl tristearate; PEG-240/HDI copolymer bis-decyltetradeceth-20 ether; PEG-100/IPDI copolymer; PEG-180laureth-50/™MG copolymer; PEG-10/lauryl dimethicone crosspolymer; PEG-15/lauryl dimethicone crosspolymer; PEG-2M; PEG-5M; PEG-7M; PEG-9M; PEG-14M; PEG-20M; PEG-23M; PEG-25M; PEG-45M; PEG-65M; PEG-90M; PEG-115M; PEG-160M; PEG-180M; PEG-120 methyl glucose trioleate; PEG-180/octoxynol-40/™MG copolymer; PEG-150 pentaerythrityl tetrastearate; PEG-4 rapeseedamide; PEG-150/stearyl alcohol/SMDI copolymer; phaseolus angularis seed powder; polianthes tuberosa extract; polyacrylate-3; polyacrylic acid; polycyclopentadiene; polyether-1; polyethylene/isopropyl maleate/MA copolyol; polyglyceryl-3 disiloxane dimethicone; polyglyceryl-3 polydimethylsiloxyethyl dimethicone; polymethacrylic acid; polyquaternium-52; polyvinyl alcohol; potassium alginate; potassium aluminum polyacrylate; potassium carbomer; potassium carrageenan; potassium chloride; potassium palmate; potassium polyacrylate; potassium sulfate; potato starch modified; PPG-2 cocamide; hydroxyethyl caprylamide; PPG-2 hydroxyethyl cocamide; PPG-2 hydroxyethyl coco/isostearamide; PPG-3 hydroxyethyl soyamide; PPG-14 laureth-60 hexyl dicarbamate; PPG-14 laureth-60 isophoryl dicarbamate; PPG-14 palmeth-60 hexyl dicarbamate; propylene glycol alginate; PVP/decene copolymer; PVP montmorillonite; Pyrus cydonia seed; Pyrus malus (apple) fiber; rhizobian gum; ricebranamide DEA; ricinoleamide DEA; ricinoleamide MEA; ricinoleamide M1PA; ricinoleamidopropyl betaine; ricinoleic acid/adipic acid/AEEA copolymer; Rosa multiflora flower wax; Sclerotium gum; sesamide DEA; sesamidopropyl betaine; sodium acrylate/acryloyldimethyl taurate copolymer; sodium acrylates/acrolein copolymer; sodium acrylates/acrylonitrogens copolymer; sodium acrylates copolymer; sodium acrylates crosspolymer; sodium acrylate/sodium acrylamidomethylpropane sulfonate copolymer; sodium acrylates/vinyl isodecanoate crosspolymer; sodium acrylate/vinyl alcohol copolymer; sodium carbomer; sodium carboxymethyl chitin; sodium carboxymethyl dextran; sodium carboxymethyl beta-glucan; sodium carboxymethyl starch; sodium carrageenan; sodium cellulose sulfate; sodium chloride; sodium cyclodextrin sulfate; sodium hydroxypropyl starch phosphate; sodium isooctylene/MA copolymer; sodium magnesium fluorosilicate; sodium oleate; sodium palmitate; sodium palm kernelate; sodium polyacrylate; sodium polyacrylate starch; sodium polyacryloyldimethyl taurate; sodium polygamma-glutamate; sodium polymethacrylate; sodium polystyrene sulfonate; sodium silicoaluminate; sodium starch octenylsuccinate; sodium stearate; sodium stearoxy PG-hydroxyethylcellulose sulfonate; sodium styrene/acrylates copolymer; sodium sulfate; sodium tallowate; sodium tauride acrylates/acrylic acid/acrylonitrogens copolymer; sodium tocopheryl phosphate; Solanum tuberosum (potato) starch; soyamide DEA; soyamidopropyl betaine; starch/acrylates/acrylamide copolymer; starch hydroxypropyltrimonium chloride; stearamide AMP; stearamide DEA; stearamide DEA-distearate; stearamide DIBA-stearate; stearamide MEA; stearamide MEA-stearate; stearamide MIPA; stearamidopropyl betaine; steareth-60 cetyl ether; steareth-100/PEG-136/HDI copolymer; stearyl alcohol; stearyl betaine; Sterculia urens gum; synthetic fluorphlogopite; tallamide DEA; tallow alcohol; tallowamide DEA; tallowamide MEA; tallowamidopropyl betaine; tallowamidopropyl hydroxysultaine; tallowamide oxide; tallow betaine; tallow dihydroxyethyl betaine; tamarindus indica seed gum; tapioca starch; TEA-alginate; TEA-carbomer; TEA-hydrochloride; trideceth-2 carboxamide MEA; tridecyl alcohol; triethylene glycol dibenzoate; trimethyl pentanol hydroxyethyl ether; Triticum vulgare (wheat) germ powder; Triticum vulgare (wheat) kernel flour; Triticum vulgare (wheat) starch; tromethamine acrylates/acrylonitrogens copolymer; tromethamine magnesium aluminum silicate; undecyl alcohol; undecylenamide DEA; undecylenamide MEA; undecylenamidopropyl betaine; welan gum; wheat germamide DEA; wheat germamidopropyl betaine; xanthan gum; yeast beta-glucan; yeast polysaccharides; Zea mays (corn) starch; and blends thereof.

In another embodiment of the present application, it is contemplated that the skin care composition of the present application can be a sunscreen composition, a dermatological composition or a cosmetic composition.

In accordance with yet another embodiment of the present application, the skin care/sunscreen compositions of the present application can exist in the form of a gel, a balm, a cream-in-gel, a balm-in-gel, a stick, a lotion, a foam, a foam cream, a cream, a moisturizer, a spray, an ointment, and a lamellar gel networks (LGNs). Other suitable and non-limiting sunscreen or skin care composition forms include: solutions, oils, solids, liquids, powders, gels, pastes, waxes, aerosols, mists, roll-ons, milks, emulsions, wipes, oil-in-water emulsion, water-in-oil emulsion, an oil-water-oil emulsion, a water-oil-water emulsion, a water-in-silicone emulsion, an oily solution, a lipid fusion, a hydro-alcoholic gel, an anhydrous gel, an aqueous gel, an alcoholic solutions or a hydro-alcoholic solution, suspensions, microemulsions, dispersions, microencapsulated products, tonics, mists, reconstitutable products, peels, soaps, mousses, glues, pomades, spritzes, putties, lacquers, serums, perms, powders, pencils, flakes, blush, highlighters, bronzers, concealers, and 2-way cake products.

According to another important embodiment of the present application, it is contemplated that the terpolymer of the present application can be employed in other non-limiting examples of personal care compositions that can comprise after-sun compositions, skin care compositions, oral care compositions, face care compositions, lip care compositions, eye care compositions, body care compositions, nail care compositions, anti-aging compositions, insect repellents, deodorant compositions, color cosmetic compositions, color-protection compositions, self-tanning compositions, and foot care compositions.

Another embodiment of the present application provides a sunscreen composition comprising: i) 0.1 wt. % to 10 wt. % of a terpolymer of vinyl pyrrolidone/acrylic acid/lauryl methacrylate having a relative viscosity of at least 3.3 cps; ii) at least one lamellar gel; iii) at least one propellant; iv) 0.1 wt. % to 60 wt. % of at least one sunscreen agent; and v) 0.1 wt. % to 20 wt. % of at least one cosmetically/dermatologically acceptable additive. Also, provided is a method for providing enhanced care to skin of humans or animals by applying the above said composition to the skin.

Yet another embodiment of the present application discloses that the pH of the composition is in the range of from about 2 to about 12. Other possible ranges of pH can be about 2 to about 4, about 4 to about 6, about 6 to about 8, about 8 to about 10, about 10 to about 12.

According to yet another embodiment, the present application discloses a method for providing enhanced care to skin of humans or animals, said method comprising topically applying to the skin of humans or animals an effective amount of a sunscreen composition comprising:
i. 0.1 wt. % to 10 wt. % of a terpolymer of vinyl pyrrolidone/acrylic acid/lauryl methacrylate having a relative viscosity of at least 3.3 cps;
ii. at least one lamellar gel;
iii. 0.1 wt. % to 60 wt. % of at least one sunscreen agent; and
iv. 0.1 wt. % to 20 wt. % of at least one cosmetically/dermatologically acceptable additive,
   wherein the care and sensory benefits are with respect to (a) providing protection from the harmful ultraviolet radiations of sun, (b) providing dry and powdery after feel to the skin, (c) providing decreased shine appearance, (d) providing enhanced water resistance of the sunscreen composition (e) providing positive appearance, (1) high integrity of shape, (a) visual compactness, (h) high spreadability, and/or (i) positive skin feel.

According to one embodiment of the present application, the skin care and sun screen compositions of the present application also impart a dry and powdery feel and decreased shine appearance on to the surface of skin of humans after their application.

Further, certain aspects of the present invention are illustrated in detail by way of the following examples. The examples are given herein for illustration of the invention and are not intended to be limiting thereof.

### EXAMPLES

The process for preparing sunscreen mousse is described in the Examples. Various ingredients including their weight proportions used for preparing the sunscreen mousse compositions are disclosed.

### Example 1: Sunscreen Mousse SPF50

| | | Example 1 |
|---|---|---|
| Phase Name | Ingredients | % W/W |
| PHASE A | D.I. water | 48.97 |
| | Tetrasodium EDTA | 0.05 |
| | Phenoxyethanol and Caprylyl | 1.00 |
| | Glycol | |
| PHASE B | Carbomer | 0.15 |
| PHASE C | Phenylbenzimidazole | 3.00 |
| | Sulfonic Acid | |
| | Purified water | 11.25 |
| | Sodium Hydroxide | 0.48 |
| PHASE D | Bis-Ethylhexyloxyphenol | 3.00 |
| | Methoxyphenyl Triazine Butyl | |
| | Methoxydibenzoylmethane | 3.00 |
| | (Avobenzone) | |
| | Ethylhexyl Salicylate | 5.00 |
| | Homosalate | 10.00 |
| | Octocrylene | 8.00 |
| | Glyceryl Stearate (and) | 4.00 |
| | Behenyl Alcohol (and) | |
| | Palmitic Acid (and) Stearic | |
| | Acid (and) Lecithin (and) | |
| | Lauryl Alcohol (and) | |
| | Myristyl Alcohol (and) Cetyl | |
| | Alcohol | |
| PHASE E | Aqua | 1.00 |
| | Sodium Hydroxide | 0.20 |
| PHASE F | VP/acrylates/lauryl | 0.90 |
| | methacrylate copolymer | |
| | | 100.00 |

### Example 2: Sensory Mousse

| Phase | | Example 2 |
|---|---|---|
| Name | Ingredients | % W/W |
| PHASE A | D.I.Waer | 76.36 |
| | Tetrasodium EDTA | 0.1 |
| | PVM/MA Decadiene | |
| | Crossoolymer | 0.2 |
| | Glycerin (and) Glyceryl | |
| | Acrylate/Acrylic Acid | |
| | Copolymer (and) | |
| | Phenoxyethanol | 5.0 |
| | Phenoxyethanol & | 1.0 |
| | Methylparaben & Ethylparaben & | |
| | Propylparaben | |
| PHASE B | Stearic Acid (and) Behenyl | 4.0 |
| | Alcohol (and) Gtyceryl Stearate | |
| | (and) Stearyl Alcohol (and) | |
| | Cetyl Alcohol (and) Palmitic | |
| | Acid (and) Hydroxyethyl | |
| | Cetearaniidopropyldimonium | |
| | Chloride | |
| | Glyceryl Dilaurate | 2.0 |
| | lsocetyl Stearate | 1.5 |
| | lsodecyl Neopentanoate | 5.0 |
| | VP/acrylates/lauryl methacrylate | 0.3 |
| | copolymer | |
| PHASE C | Water/Aqua | 1.0 |
| | Sodium hydroxide | 0.04 |
| PHASE D | Disodium | |
| | Lauriminodipropionate | |
| | TocooheNI Phosohates | 3.0 |
| | Violet 2 hydro K7116-J | 0.2 |
| | RED40 | 0.3 |
| | | 100.00 |

### Example 3: Sensory Mousse

| | | Example 3 |
|---|---|---|
| Phase Name | Ingredients | % W/W |
| PHASE A | D.I water | 81.79 |
| | Tetrasodium EDTA | 0.05 |
| | Phenoxyethanol (and) Caprylyl | 0.90 |
| | glycol | |
| | Phenoxyethanol & Benzoic | 0.40 |
| | Acid & | |
| | Dehydroacetic Acid | |
| PHASE B | Carbomer | 0.25 |
| PHASE C | Glyceryl Stearate & Behenyl | 3.50 |
| | Alcohol & Palmitic Acid & | |
| | Stearic Acid & Lecithin & Lauryl | |
| | Alcohol & Myristyl Alcohol & | |
| | Cetyl Alcohol | |
| | Butyrospermum Parkii (Shea) | 2.00 |
| | Butter | |
| | lsocetyl Stearate | 3.00 |
| | lsodecyl Neopentanoate | 6.00 |
| | VP/acrylates/lauryl methacrylate | 0.70 |
| | copolymer | |
| PHASE D | Water/Aqua | 1.00 |
| | Sodium hydroxide | 0.11 |
| PHASE E | red 40 | 0.30 |
| | | 100.00 |

### Example 4: Sensory Mousse

| | | Example 4 |
|---|---|---|
| Phase Name | Ingredients | % W/W |
| PHASE A | .D.I water | 82.00 |
| | Tetrasodium EDTA | 0.05 |
| | Phenoxyethanol (and) | 0.90 |
| | Caprylyl glycol | |
| | Phenoxyethanol & | 0.40 |
| | Benzoic Acid & | |
| | Dehydroacetic Acid | |
| PHASE B | Carbomer | 0.25 |
| PHASE C | Glyceryl Stearate & | 3.50 |
| | Behenyl Alcohol & | |
| | Palmitic Acid & Stearic | |
| | Acid & Lecithin & Lauryl | |
| | Alcohol & Myristyl | |
| | Alcohol & Cetyl Alcohol | |
| | Butyrospermum Parkii | 2.00 |
| | (Shea) Butter | |
| | lsocetyl Stearate | 2.00 |
| | lsodecyl Neopentanoate | 7.00 |
| | VP/acrylates/lauryl | 0.50 |
| | methacrylate copolymer | |
| PHASE D | Purified water | 1.00 |
| | Sodium hydroxide | 0.10 |
| PHASE E | red 40 | 0.30 |
| | | 100.00 |

### Example 5: Sensory Mousse

| | | Example 5 |
|---|---|---|
| Phase Name | Ingredients | % W/W |
| PHASE A | D.I water | 64.78 |
| | Tetrasodium EDTA | 0.05 |
| | Phenoxyethanol (and) Caprylyl | 1.00 |
| | glycol | |
| | Methylparaben | 0.30 |
| PHASE B | Carbomer | 0.10 |
| PHASE C | Glyceryl Stearate & Behenyl | 4.00 |
| | Alcohol & Palmitic Acid & | |
| | Stearic Acid & Lecithin & | |
| | Lauryl Alcohol & Myristyl | |
| | Alcohol & Cetyl Alcohol | |
| | Ceteareth 20 | 0.10 |
| | Avobenzone | 3.00 |
| | Octisalate | 5.00 |
| | Octocrylene | 4.00 |
| | Homosalate | 10.00 |
| | Diisopropyl Adipate | 2.00 |
| | Propylparaben | 0.15 |
| PHASE D | Purified water | 1.00 |
| | Sodium hydroxide | 0.12 |
| PHASE E | VP/acrylates/lauryl | 1.00 |
| | methacrylate copolymer | |
| | Phenoxyethanol (and) Benzoic | 0.40 |
| | Acid (and) Dehydroacetic Acid | |
| | Cyclomethicone | 3.00 |
| | | 100.00 |

### Example 6: Mousse with Carbon Dioxide

| | | Example 6 |
|---|---|---|
| Phase | Ingredients | % W/W |
| PHASE A | D.I Water | 63.71 |
| | Disodium EDTA | 0.05 |
| | Phenoxyethanol (and) | 0.98 |
| | Caprylyl Glycol | |
| | Methylparaben | 0.30 |
| | Carbomer | 0.10 |
| PHASE B | Ceteareth-20 | 0.10 |
| | Avobenzone | 2.94 |
| | Ethylhexyl Salicylate | 4.90 |
| | Octocrylene | 3.92 |
| | Homosalate | 9.80 |
| | Diisopropyl Adipate | 1.96 |
| | Propylparaben | 0.15 |
| | Glyceryl stearate (and) | 3.92 |
| | behenyl alcohol (and) | |
| | palmitic acid | |
| | (and)stearic aid | |
| | (and)lecithin | |
| | (and)lauryl alcohol | |
| | (and) myristyl alcohol | |
| | (and) cetyl alcohol | |
| PHASE C | Sodium hydroxide | 1.17 |
| | 10% | |
| PHASE D | VP/Acrylate/Lauryl | 0.98 |
| | methacrylate | |
| | Copolymer | |
| | Cyclopentasiloxane | 2.94 |
| | Phenoxyethanol (and) | 0.38 |
| | Benzoic acid (and) | |
| | dehydroacetic acid | |
| PHASE E | Carbon dioxide | 1.70 |
| | Total | 100.00 |

### Example 7 and 8: Mousse with Nitrous Oxide and Nitrogen

| | | Example | Example |
|---|---|---|---|
| | | 7 | 8 |
| | Ingredients | % w/w | % w/w |
| PHASE A | D.I Water | 63.71 | 63.71 |
| | Disodium EDTA | 0.05 | 0.05 |
| | Phenoxyethanol (and) Caprylyl Glycol | 0.98 | 0.98 |
| | Methylparaben | 0.30 | 0.30 |
| | Carbomer | 0.10 | 0.10 |
| PASE B | Ceteareth-20 | 0.10 | 0.10 |
| | Avobenzone | 2.94 | 2.94 |
| | Ethylhexyl Salicylate | 4.90 | 4.90 |
| | Octocrylene | 3.92 | 3.92 |
| | Homosalate | 9.80 | 9.80 |
| | Diisopropyl Adipate | 1.96 | 1.96 |
| | Propylparaben | 0.15 | 0.15 |
| | Glyceryl stearate (and) behenyl | 3.92 | 3.92 |
| | alcohol (and) palmitic acid (and)stearic | | |
| | aid (and)lecithin (and)lauryl | | |
| | alcohol (and) myristyl alcohol | | |
| | (and) cetyl alcohol | | |
| PHASE C | Sodium hydroxide 10% | 1.17 | 1.17 |
| PHASE D | VP/Acrylate/Lauryl methacrylate | 0.98 | 0.98 |
| | Copolymer | | |
| | Cyclopentasiloxane | 2.94 | 2.94 |
| | Phenoxyethanol (and) Benzoic | 0.38 | 0.38 |
| | acid (and) dehydroacetic acid | | |
| PHASE E | Nitrous Oxide | 1.70 | / |
| | Nitrogen | / | 1.70 |
| | Total | 100.00 | 100.00 |

### Example 9, 10A, 10B and 10C: Mousse with Isobutane and Propane/Isobutane

| | | Example 9 | Example 10A | Example 10B | Example 10C |
|---|---|---|---|---|---|
| | Ingredients | % w/w | % w/w | % w/w | % w/w |
| PHASE A | D.I. Water | 58.15 | 58.15 | 58.15 | 58.15 |
| | Disodium EDTA | 0.05 | 0.05 | 0.05 | 0.05 |
| | Phenoxyethanol | 0.90 | 0.90 | 0.90 | 0.90 |
| | (and) Caprylyl Glycol | | | | |
| | Methylparaben | 0.27 | 0.27 | 0.27 | 0.27 |
| | Carbomer | 0.10 | 0.10 | 0.10 | 0.10 |
| PHASE B | Ceteareth-20 | 0.10 | 0.10 | 0.10 | 0.10 |
| | Avobenzone | 2.70 | 2.70 | 2.70 | 2.70 |
| | Ethylhexyl Salicylate | 4.50 | 4.50 | 4.50 | 4.50 |
| | Octocrylene | 3.60 | 3.60 | 3.60 | 3.60 |
| | Homosalate | 9.00 | 9.00 | 9.00 | 9.00 |
| | Diisopropyl Adipate | 1.80 | 1.80 | 1.80 | 1.80 |
| | Propylparaben | 0.15 | 0.15 | 0.15 | 0.15 |
| | Glyceryl stearate | 3.60 | 3.60 | 3.60 | 3.60 |
| | (and) behenyl alcohol | | | | |
| | (and) palmitic acid | | | | |
| | (and)stearic aid | | | | |
| | (and)lecithin | | | | |
| | (and)lauryl alcohol | | | | |
| | (and) myristyl | | | | |
| | alcohol (and) cetyl alcohol | | | | |
| PHASE C | Sodium hydroxide | 1.08 | 1.08 | 1.08 | 1.08 |
| | 10% | | | | |
| PHASE D | VP/Acrylate/Lauryl | 0.90 | 0.90 | 0.90 | 0.90 |
| | methacrylate | | | | |
| | Copolymer | | | | |
| | Cyclopentasiloxane | 2.70 | 2.70 | 2.70 | 2.70 |
| | Phenoxyethanol | 0.40 | 0.40 | 0.40 | 0.40 |
| | (and) Benzoic acid | | | | |
| | (and) dehydroacetic acid | | | | |
| PHASE E | Isobutane | 10.00 | / | / | / |
| | Propane/Isobutane | / | 10.00 | / | / |
| | (A-40) | | | | |
| | Propane/Isobutane | / | / | 10.00 | / |
| | (A-46) | | | | |
| | Propane/Isobutane | / | / | / | 10.00 |
| | (A-70) | | | | |
| | | 100.00 | 100.00 | 100.00 | 100.00 |

A-40, A-46 and A-70 are blends of hydrocarbon propellants commonly used in aerosol products. The “A” is for aerosol grade and the number is the vapor pressure of the blend (psig) at 70° F. (21.1° C.). The gasses blended to achieve this pressure are as listed, isobutane (31 psig) and propane (108 psig). The gauge used to measure these pressures subtracts the 14.7 psig (absolute) of the atmosphere.

### Example 11, 12, 13 and 14: Mousse with Dimethyl Ether, 1,1-Difluoroethane, Tetrafluoropropene and n-Butane

| | | Example 11 | Example 12 | Example 13 | Example 14 |
|---|---|---|---|---|---|
| | Ingredients | % w/w | % w/w | % w/w | % w/w |
| PHASE A | Water | 58.15 | 58.15 | 58.15 | 58.15 |
| | Disodium EDTA | 0.05 | 0.05 | 0.05 | 0.05 |
| | Phenoxyethanol (and) | 0.90 | 0.90 | 0.90 | 0.90 |
| | Caprylyl Glycol | | | | |
| | Methylparaben | 0.27 | 0.27 | 0.27 | 0.27 |
| | Carbomer | 0.10 | 0.10 | 0.10 | 0.10 |
| PHASE B | Ceteareth-20 | 0.10 | 0.10 | 0.10 | 0.10 |
| | Avobenzone | 2.70 | 2.70 | 2.70 | 2.70 |
| | Ethylhexyl Salicylate | 4.50 | 4.50 | 4.50 | 4.50 |
| | Octocrylene | 3.60 | 3.60 | 3.60 | 3.60 |
| | Homosalate | 9.00 | 9.00 | 9.00 | 9.00 |
| | Diisopropyl Adipate | 1.80 | 1.80 | 1.80 | 1.80 |
| | Propylparaben | 0.15 | 0.15 | 0.15 | 0.15 |
| | Glyceryl stearate (and) | 3.60 | 3.60 | 3.60 | 3.60 |
| | behenyl alcohol (and) | | | | |
| | palmitic acid | | | | |
| | (and)stearic aid | | | | |
| | (and)lecithin | | | | |
| | (and)lauryl alcohol | | | | |
| | (and) myristyl alcohol | | | | |
| | (and) cetyl alcohol | | | | |
| PHASE C | Sodium hydroxide 10% | 1.08 | 1.08 | 1.08 | 1.08 |
| PHASE D | VP/Acrylate/Lauryl | 0.90 | 0.90 | 0.90 | 0.90 |
| | methacrylate | | | | |
| | Copolymer | | | | |
| | Cyclopentasiloxane | 2.70 | 2.70 | 2.70 | 2.70 |
| | Phenoxyethanol (and) | 0.40 | 0.40 | 0.40 | 0.40 |
| | Benzoic acid (and) | | | | |
| | dehydroacetic acid | | | | |
| PHASE E | Dimethyl Ether | 10.00 | / | / | / |
| | 1,1-Difluoroethane | / | 10.00 | / | / |
| | Tetrafluoropropene | / | / | 10.00 | / |
| | n-Butane | / | / | / | 10.00 |
| | | 100.00 | 100.00 | 100.00 | 100.00 |

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of this invention.

What is claimed is:

1. A personal care composition, comprising:
i. 0.1 wt. % to 10 wt. % of a terpolymer of vinyl pyrrolidone/acrylic acid/lauryl methacrylate having a relative viscosity of at least 3.3 cps; and
ii. at least one lamellar gel.

2. The personal care composition according to claim 1, wherein the lamellar gel is a blend of:
a) one or more fatty alcohols;
b) one or more fatty acids and/or their esters;
c) optionally one or more low molecular weight alcohols;
d) optionally esterified oils; and
e) optionally cationic swellants.

3. The personal care composition according to claim 1, wherein the lamellar gel is a blend of:
a) one or more C₈-C₂₄ fatty alcohols;
b) one or more C₈-C₂₄ fatty acids and/or their esters;
c) optionally a C₂-C₁₀ low molecular weight alcohols;
d) optionally esterified oils; and
e) optionally cationic swellants.

4. The personal care composition according to claim 3, wherein the C₈-C₂₄ fatty alcohol is selected from the group consisting of behenyl alcohol, cetyl alcohol, stearyl alcohol, lauryl alcohol, myristyl alcohol and a mixture thereof.

5. The personal care composition according to claim 3, wherein the C₈-C₂₄ fatty acid is selected from the group consisting of stearic acid, palmitic acid and a mixture thereof.

6. The personal care composition according to claim 3, wherein the C₈-C₂₄ fatty acid ester is selected from the group consisting of glyceryl stearate, sorbitan monostearate, ricinoleyl monomaleate, PEG-100 stearate, triglycerides and a mixture thereof.

7. The personal care composition according to claim 3, wherein the C₂-C₁₀ low molecular weight alcohol is selected from the group consisting of propylene glycol, butylene glycol, isopropyl alcohol and a mixture thereof.

8. The personal care composition according to claim 3, wherein the esterified oil is a maleated soybean oil.

9. The personal care composition according to claim 3, wherein the cationic swellant is a saturated fatty acid adduct of amidopropyldimethyl-2-hydroxy ethyl ammonium chloride.

10. The personal care composition according to claim 1, wherein the lamellar gel is present in an amount of about 1.00% w/w to about 20.00% w/w of the personal care composition.

11. The personal care composition according to claim 1, further comprising at least one propellant selected from the group consisting of CO₂, propane, butane, isobutane, dimethyl ether, nitrous oxide, nitrogen, oxygen, air, isopentane, hydrofluoroolefin, and a mixture thereof.

12. The personal care composition according to claim 11, wherein the propellant is present in an amount of about 1.00% w/w to about 20.00% w/w.

13. The personal care composition according to claim 1, wherein the personal care composition further comprises at least one cosmetically/dermatologically acceptable additive.

14. The personal care composition according to claim 13, wherein the cosmetically/dermatologically acceptable additive is selected from the group consisting of diluents, emollients, humectants, thickeners, preservatives, coloring agents, sunscreen agents, moisturizing agents, film formers/waterproofing agents, bio-active ingredients, pharmaceutical ingredients, pH adjusting agents, chelating agents, rheology modifying agents, fragrance agents, plant extracts, absorbents, vitamins, photo stabilizing agents, sunscreen boosters, anti-oxidants, exfoliating agents, liquid carriers, waxes, conditioners, lubricants, anti-bacterial agents, anti-aging agents, insect repellants, and combinations thereof.

15. The personal care composition according to claim 14, wherein the insect repellant is selected from the group consisting of N,N-diethyl m-toluamide (DEET), p-menthane-3,8-diol, permethrin, allethrin, piperonyl butoxide, lemongrass oil, citronella oil, eucalyptus oil, camphor, geranium oil, ethyl hexanediol, ethyl butylacetylaminopropionate, and hydroxyethyl-isobutyl-piperidine.

16. The personal care composition according to claim 1, wherein the personal care composition is suitable for application to the skin, hair, lips, lashes and nails.

17. The personal care composition according to claim 1, wherein the personal care composition is in the form of gels, balms, cream-in-gels, balm-in-gels, sticks, lotions, foams, foam creams, creams, moisturizers, sprays, ointments, mousses and a lamellar gel networks (LGNs).

18. The personal care composition according to claim 1, wherein the personal care composition is a sun screen composition or a cosmetic composition.

19. The personal care composition according to claim 1, wherein the weight average molecular weight of the terpolymer ranges from about 150,000 to about 300,000 Daltons.

20. A sunscreen composition comprising:
i. 0.1 wt. % to 10 wt. % of a terpolymer of vinyl pyrrolidone/acrylic acid/lauryl methacrylate having a relative viscosity of at least 3.3 cps;
ii. at least one lamellar gel;
iii. 0.1 wt. % to 60 wt. % of at least one sunscreen agent; and
iv. 0.1 wt. % to 20 wt. % of at least one cosmetically/dermatologically acceptable additive.

21. The sunscreen composition according to claim 20, wherein the sunscreen agent is selected from the group consisting of methoxydibenzoylmethane; octyl salicylate; pentyl dimethyl PABA; octyl dimethyl PABA; benzophenone-1; benzophenone-6; 2-(2H-benzotriazole-2-yl)-4,6-di-tert-pentylphenol; ethyl-2-cyano-3,3-diphenylacrylate; homomethyl salicylate (homosalate); bis-ethylhexyloxyphenol methoxyphenyl triazine; methyl-(1,2,2,6,6-pentamethyl-4-piperidyl)-sebacate; 2-(2H-benzotriazole-2-yl)-4-methylphenol; diethylhexyl butamido triazone; amyl dimethyl PABA; 4,6-bis(octylthiomethyl)-o-cresol; red petroleum; ethylhexyl triazone; octocrylene; isoamyl-p-methoxycinnamate; drometrizole; titanium dioxide; 2,4-di-tert-butyl-6-(5-chloro-2H-benzotriazole-2-yl)-phenol; 2-hydroxy-4-octyloxybenzophenone; benzophenone-2; diisopropyl methylcinnamate; PEG-25 PABA; 2-(1,1-dimethylethyl)-6-[[3-(1,1-demethylethyl)-2-hydroxy-5-methylphenyl]methyl-4-methylphenyl acrylate; drometrizole trisiloxane; menthyl anthranilate; butyl methoxydibenzoylmethane; 2-ethoxyethyl p-methoxycinnamate; benzylidene camphor sulfonic acid; dimethoxyphenyl-[1-(3,4)]-4,4-dimethyl 1,3-pentanedione; zinc oxide; N,N′-hexane-1,6-diylbis[3-(3,5-di-tert-butyl-4-hydroxyphenylpropionamide)]; pentaerythritol tetrakis[3-(3,5-di-tert-butyl-4-hydroxyphenyl)propionate]; 2,6-di-tert-butyl-4-[4,6-bis(octylthio)-1,3,5-triazin-2-ylamino]phenol; 2-(2H-benzotriazole-2-yl)-4,6-bis(1-methyl-1-phenylethyl)phenol; trolamine salicylate; diethylanolamine p-methoxycinnamate; polysilicone-15; 4-methylbenzylidene camphor; bisoctrizole; N-phenyl-benzenamine; reaction products with 2,4,4-trimethylpentene; sulisobenzone; (2-ethylhexyl)-2-cyano-3,3-diphenylacrylate; digalloyl trioleate; polyacrylamido methylbenzylidene camphor; glyceryl ethylhexanoate dimethoxycinnamate; 1,3-bis-[(2′-cyano-3′,3′-diphenylacryloyl)oxy]-2,2-bis-[(2′-cyano-bis-(2,2,6,6-tetramethyl-4-piperidyl)-sebacate benzophenone-5; 1,3,5-tris(3,5-di-tert-butyl-4-hydroxybenzyl)-1,3,5-triazine-2,4,6(1H,3H,5H)-trione; phenol, 2,2′-[6-(4-methoxyphenyl)-1,3,5-triazine-2,4-diyl]bis(5-(2-ethylhexyl)oxy); hexamethylendiamine; benzophenone-8; ethyl-4-bis(hydroxypropyl)aminobenzoate; 6-tert-butyl-2-(5-chloro-2H-benzotriazole-2-yl)-4-methylphenol; p-aminobenzoic acid; 3,3′,3″,5,5′,5″-hexa-tert-butyl-a-a′-a″-(mesitylene-2,4,6-triyl)tri-p-cresol; lawsone with dihydroxyacetone; benzophenone-9; benzophenone-4; ethylhexyl dimethoxy benzylidene dioxoimidazoline propionate; N,N′-bisformyl-N,N′-bis-(2,2,6,6-tetramethyl-4-piperidinyl)-; 3-benzylidene camphor; terephthalylidene dicamphor sulfonic acid; camphor benzalkonium methosulfate; bisdisulizole disodium; etocrylene; ferulic acid; 2-(2H-benzotriazole-2-yl)-4-(1,1,3,3-tetramethylbutyl)-phenol; 4,6-bis(dodecylthiomethyl)-o-cresol; β-2-glucopyranoxy propyl hydroxy benzophenone; phenylbenzimidazole sulfonic acid; benzophenone-3; diethylamine hydroxybenzoyl hexylbenzoate; 3′,3′-diphenylacryloyl)oxy]methyl}-propane; ethylhexyl p-methoxycinnamate, and blends thereof.

22. The sunscreen composition according to claim 20, having a sun protection factor of at least 10.

23. The sunscreen composition according to claim 20, wherein the pH of the composition is in the range of from about 2 to about 12.

24. A method for providing enhanced care and sensory benefits to skin of humans or animals, said method comprising topically applying to the skin of humans or animals an effective amount of a sunscreen composition comprising:
i. 0.1 wt. % to 10 wt. % of a terpolymer of vinyl pyrrolidone/acrylic acid/laurel methacrylate having a relative viscosity of at least 3.3 cps;
ii. at least one lamellar gel;
iii. 0.1 wt. % to 60 wt. % of at least one sunscreen agent; and
iv. 0.1 wt. % to 20 wt. % of at least one cosmetically/dermatologically acceptable additive,
wherein the care and sensory benefits are with respect to (a) providing protection from the harmful ultraviolet radiations of sun, (b) providing dry and powdery after feel to the skin, (c) providing decreased shine appearance, (d) providing enhanced water resistance of the sunscreen composition (e) providing positive appearance, (f) high integrity of shape, (g) visual compactness, (h) high spreadability, and/or (i) positive skin feel.
